(19) United States
(12) Patent Application Publication
(10) Pub. No.: US 2006/0147957 A1
(43) Pub. Date: Jul. 6, 2006

(54) Methods for high throughput sample preparation for microarray analysis

(75) Inventors: Zuwei Qian, Fremont, CA (US); Thomas B. Ryder, Los Gatos, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/273,963

(22) Filed: Nov. 14, 2005

Related U.S. Application Data
(60) Provisional application No. 60/627,211, filed on Nov. 12, 2004.

(51) Int. Cl. C12Q 1/68

(52) U.S. Cl. 435/6

(57) ABSTRACT

Automated methods for sample preparation for amplification of nucleic acid samples to prepare target for hybridization to microarrays are disclosed. Automated methods for hybridizing target to microarrays, washing and staining microarrays are also disclosed. Improved conditions for hybridization and for storage and scanning of arrays with hybridized target are also disclosed.

### RELATED APPLICATIONS

This application claims priority to U.S. Provisional Application Ser. No. 60/627,211 filed on Nov. 12, 2004, which is incorporated herein by reference in its entirety for all purposes.

### FIELD OF THE INVENTION

Methods and reagents for high throughput analysis of nucleic acids on high density microarrays are disclosed. Methods include automated target prep methods and automated hybridization, washing, and staining methods.

### BACKGROUND OF THE INVENTION

Nucleic acid sample preparation methods have greatly transformed laboratory research that utilize molecular biology and recombinant DNA techniques and have also impacted the fields of diagnostics, forensics, nucleic acid analysis and gene expression monitoring, to name a few. There remains a need in the art for methods for reproducibly and efficiently fragmenting nucleic acids used for hybridization to oligonucleotide arrays.

### SUMMARY OF THE INVENTION

Automated methods for preparing amplified target for hybridization to microrarrys are disclosed. Methods and reagents for high throughput processing of microarrays are also disclosed, including buffer compositions for washing and storing arrays after hybridization.

### BRIEF DESCRIPTION OF THE DRAWINGS

FIG. 1 is a schematic of steps in the clean-up and elution of the cDNA. Individual steps are numbered from 10 to 23.

FIG. 2 is a schematic of steps in the clean-up and elution of the cRNA after IVT. Individual steps are numbered from 26 to 40.

### DETAILED DESCRIPTION OF THE INVENTION

a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H.A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), Ser. No. 09/910,292 (U.S. Patent Application Publication 20030082543), and Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2 ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication Number 20020183936), 10/065,856, 10/065,868, 10/328,818, 10/328,872, 10/423,403, and 60/482,389.

b) Definitions

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “array plate” as used herein refers to a body having a plurality of arrays in which each microarray is separated by a physical barrier resistant to the passage of liquids and forming an area or space, referred to as a well, capable of containing liquids in contact with the probe array.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a bioploymer is a “biomonomer”.

The term “cartridge” as used herein refers to a body forming an area or space referred to as a well wherein a microarray is contained and separated from the passage of liquids.

The term “clamping plate” as used herein refers to a device used for fastening two or more parts.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “effective amount” as used herein refers to an amount sufficient to induce a desired result.

The term “excitation energy” as used herein refers to energy used to energize a detectable label for detection, for example illuminating a fluorescent label. Devices for this use include coherent light or non coherent light, such as lasers, UV light, light emitting diodes, an incandescent light source, or any other light or other electromagnetic source of energy having a wavelength in the excitation band of an excitable label, or capable of providing detectable transmitted, reflective, or diffused radiation.

The term “gaskets or o-ring” as used herein refers to any of a wide variety of seals or packings used between joined parts to prevent the escape of a gas or fluid. Gaskets or o-rings can be made of materials such as elastomer.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “glue manifold” as used herein refers to an adhesive dispensing device. A glue manifold is a specifically designed fluidic device that can distribute the adhesive through channels to a receptacle plate similar to the array plate format.

The term “glue stamper” as used herein refers to a plate which is used for controlled application of adhesive to an array plate. The glue stamper could be made of any suitable materials such as an elastomer.

The term “holding plate” as used herein refers to a body for temporal placing of a set of arrays before they are connected to a well plate.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics.

The term “label” as used herein refers to a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “linkage disequilibrium” or sometimes refer by allelic association as used herein refers to the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

The term “microtiter plates” as used herein refers to arrays of discrete wells that come in standard formats (96, 384 and 1536 wells) which are used for examination of the physical, chemical or biological characteristics of a quantity of samples in parallel.

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “optically clear” as used herein refers to the property of a material for transmitting light waves with a minimum loss of intensity or attenuation of the light.

The term “pick up plate” as used herein refers to a device to perform a transfer of arrays into an array plate for example with the use of suction.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “reader” or “plate reader” as used herein refers to a device which is used to identify hybridization events on an array, such as the hybridization between a nucleic acid probe on the array and a fluorescently labeled target. Readers are known in the art and are commercially available through Affymetrix, Santa Clara Calif. and other companies. Generally, they involve the use of an excitation energy (such as a laser) to illuminate a fluorescently labeled target nucleic acid that has hybridized to the probe. Then, the reemitted radiation (at a different wavelength than the excitation energy) is detected using devices such as a CCD, PMT, photodiode, or similar devices to register the collected emissions. See U.S. Pat. No. 6,225,625.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “surface” or “active probe surface” or “target surface” as used herein refers to the area of the microarray to be analyzed with reagents.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

The term “wafer” as used herein refers to a substrate having surface to which a plurality of arrays are bound. In a preferred embodiment, the arrays are synthesized on the surface of the substrate to create multiple arrays that are physically separate. In one preferred embodiment of a wafer, the arrays are physically separated by a distance of at least about 0.1, 0.25, 0.5, 1 or 1.5 millimeters. The arrays that are on the wafer may be identical, each one may be different, or there may be some combination thereof. Particularly preferred wafers are about 8″×8″ and are made using the photolithographic process.

The term “well plate” as used herein refers to a body with a plurality of cavities open at both wherein the cavities form an area or space referred to as a well wherein each well will hold an array.

c) Description

High throughput automated and semi-automated methods for preparing nucleic acid sample for hybridization to a microarray and for performing hybridization of target, washing and staining microarrays are disclosed. Reaction conditions and buffers optimized for automated methods are also disclosed. In many of the embodiments the methods are optimized for use on a liquid handling robot, such as the Biomek FX system (Beckman/Coulter) or the Cyclone system (Calliper). Robotic arms move over the station and aspirate and dispense liquid from various stations into microplates. The work surface may be customized by the addition of automated functionalities, for example, shaking, stirring, heating, cooling, and thermal cycling equipment.

In a preferred embodiment sample preparation is automated. Sample preparation in many embodiments includes the generation of a labeled nucleic acid sample suitable for hybridization to a microarray from a nucleic acid sample obtained from a biological source. The starting sample may include, for example, total RNA, polyA RNA, genomic DNA, polyA minus RNA, cDNA or cRNA.

Many methods of sample preparation may be automated using the disclosed methods. In one embodiment the nucleic acid sample is total RNA or a subset of total RNA, for example, polyA RNA, or total RNA that may be enriched for targets of interest by, for example, depletion of one or more RNAs, for example rRNA may be depleted or globin mRNAs may be depleted (for methods of depletion see U.S. patent application Ser. No. 10/684,205 and U.S. Pat. No. 6,613,516.). RNA may be amplified, for example, by reverse transcription using an oligo dT-T7 promoter primer as described in the Affymetrix Expression Analysis Technical Manual and in U.S. Pat. Nos. 5,545,522, 6,794,138, and 6,582,906. Other methods of amplification may include use of random primers to prime first strand cDNA synthesis from RNA or DNA. In some embodiments the assay is the GeneChip Whole Transcript (WT) sense target labeling assay as described in the assay manual (PN 701880 Rev. 2) from Affymetrix. Random primers with a universal 5′ region may be used in amplification. The universal region may include, for example, a RNA polymerase promoter region, such as a T7 promoter, or a universal priming site that can be used in a subsequent round of amplification, such as PCR or strand displacement amplification. For examples of amplification methods see U.S. Patent Pubs. 20040209298 and 20040214210 and U.S. patent application Ser. Nos. 10/917,643 and 60/550,368.

In a preferred embodiment automated target prep includes the following steps: primer annealing, first strand cDNA synthesis, second strand cDNA synthesis, T4 polymerase synthesis, cDNA purification, wash and elution, IVT reaction, and cRNA purification, washing and elution. These steps may be followed with automated cRNA quantization and normalization followed by fragmentation. A detailed description of the applicable methods is also provided in the GENECHIP Expression Analysis Technical Manual for Cartridge Arrays Using the GeneChip Array Station (P/N 702064 Rev. 1) and in the Affymetrix GENECHIP Array Station User's Guide (2005), both available from Affymetrix (P/N 701859 Rev. 2, September 2005).

In some embodiments the double stranded cDNA is purified after synthesis using magnetic bead technology as described in U.S. Pat. Nos. 5,898,071 and 5,705,628. Briefly, nucleic acids may be separated from a solution by reversibly and non-specifically binding the polynucleotides to a solid surface, such as a magnetic microparticle or bead, having a functional group-coated surface. The salt and polyalkylene glycol concentration of the solution is adjusted to allow binding of the polynucleotide to the magnetic particles. The magnetic particles are separated from the solution and the polynucleotides are eluted from the magnetic microparticles. This technology is available from Agencourt Bioscience Corp. as products for Solid Phase Reversible Immobilization (SPRI). In a preferred embodiment carboxylate-modified polymer coated magnetic beads with a polystyrene core are used.

In another embodiment hybridization, washing and staining are automated. Generally the steps involved are pre-hybridization of the arrays, the arrays are on pegs arranged in a multiwell format and hybridization is performed using a hybridization tray that holds the solutions. Pre hybridization is followed by hybridization of the sample to the array in a hybridization solution. This is followed by low stringency washing steps, high stringency washing steps, staining steps and storage of the array in a buffer suitable for scanning. The peg plate may be moved from hybe tray to each wash tray. The peg plate may be dipped into each tray a specified number of times.

In one embodiment human intervention is required at only three points after the beginning of the automated sample prep and prior to the completion of scanning: a first intervention between sample preparation and hybridization, a second intervention between hybridization and washing/staining and a third intervention between washing/staining and scanning. In this embodiment no interventions are required during scanning, or during washing and staining or during scanning.

In one embodiment reagents are loaded into wellplates manually prior to the start of sample preparation and prior to the hybridization, washing and staining procedure.

In a preferred embodiment the following buffers may be used for hybridization, washing and staining. Pre hybridization buffer of about 100 mM MES, 1M NaCl, 20 mM EDTA, 0.01% Tween 20, and 2.5 M TMACL. TMACL may be present in the disclosed buffer mixtures at about 1-2M, about 2-3M, about 3-4M or about 4-5M.

In a preferred embodiment the hybridization buffer is about 100 mM MES, 1M NaCl, 20 mM EDTA, 0.01% Tween 20, 0.1 mg/ml Herring sperm DNA (Promega), 0.5 mg/ml Acetylated BSA, (Invitrogen), 10% DMSO (Sigma), 6 mM PVP (Poly Sciences), Denhardt's Solution, Human Cot-1, and TMAC. The total volume is 300 μl including the sample.

Low stringency wash may be 6× SSPE, 0.01% Tween-20. High stringency wash may be 68 mM MES, 0.1 M NaCl, 0.01% Tween-20. Incubation for high stringency wash may be in a volume of about 85 μl wash for about 25 minutes at about 41° C. Pegs may be rinsed in low stringency wash before staining. To wash the peg plate may be moved to a first tray containing 6× SSPE, 0.01% tween-20 and dipped into the tray about 36 times, then moved to a second plate and the 36 dips repeated and then repeat the same with a third and fourth tray. Each of the 4 trays contains the 6× SSPE, 0.01% tween-20 low stringency wash buffer. The peg plate is then placed in the second stain tray and incubated for 10 min at room temp. The washing in low stringency wash (LSW) buffer with 36 dips in each of 4 trays containing LSW may be repeated. The peg plate may be moved to a tray containing the third stain and incubated for 10 min at room temp. After the third stain the peg plate may be washed again with the LSW, 4 trays with 36 dips per tray. The washed peg plate may then be stored in a tray containing about 70 μl of MES holding buffer in each well. MES buffer is 68 mM MES, 0.1 M NaCl and 0.01% Tween-20.

For staining the peg plate may be moved into at tray containing the first stain solution and incubated at room temp for 10 min.

SAPE solution is 12×MES, 5M NaCl, 10% Tween 20, 50 mg/ml Acetylated BSA, 1 mg/ml SAPE, 20× SSPE, and 50× Denhardt's Solution.

Antibody solution is 12×MES, 5 M NaCl, 10% Tween 20, 50 mg/ml Acetylated BSA, Goat IgG, 10 mg/ml in PBS, 0.5 mg/ml Biotinylated antibody, 20× SSPE, and 50× Denhardt's Solution. Non stringent wash is 20× SSPE and 10% Tween-20. Stringent Wash is 100 mM MES, 20× SSPE, 100 mM NaCl, and 10% Tween 20. Array holding buffer is 12× MES, 5 M NaCl, and 10% Tween 20.

Pre hybe may be 100 mM Mes, 1 M NaCl, 20 mM EDTA, and 0.01% Tween 20. Hybe may be 100 mM MES, 1 M NaCl, 20 mM EDTA, 0.01% Tween 20, 0.1 mg/ml herring sperm DNA, 0.5 mg/ml BSA, 10% DMSO and 6 mM PVP or 100 mM MES, 1 M NaCl, 20 mM EDTA, 0.01% Tween 20, 0.11 mg/ml herring sperm DNA, 0.556 mg/ml BSA, and 2.5 M TMAC or 0.56 M MES, 0.0115% Tween 20, 0.115 mg/ml herring sperm DNA, 5% DMS), 5.77 mM EDTA, 2.5× Denhardt's, 11.5 μg/ml human cot-1 and 2.69 M TMAC for Mapping arrays. Array holding buffer is 100 mM MES, 1 M NaCl and 0.01% tween-20, or 68 mM MES, 1 M NaCl and 0.01% tween-20. Stringent wash may be 100 mM MES, 0.1 M NaCl and 0.01% Tween 20, or 68 mM MES, 0.1 M NaCl and 0.01% Tween 20 or 0.6× SSPE and 0.01% Tween 20 for Mapping arrays. Non-stringent wash may be 6× SSPE, 0.01% Tween 20. Antibody solution may be 100 mM MES, 1 M NaCl, 0.05% Tween 20, 2 mg/ml BSA, 0.1 mg/ml goat IgG, and 3 μg/ml biotinylated antibody or for Mapping arrays it may be 5 μg/ml biotinylated antibody, 6× SSPE and 1× Denhardt's solution. SAPE solution may be 100 mM MES, 1 M NaCl, 0.05% Tween 20, 2 mg/ml BSA, and 10 μg/mL SAPE or 0.01% Tween 20, 10 μg/ml SAPE, 6× SSPE and 1× Denhardt's for Mapping arrays.

To determine optimal conditions for cRNA labeling by RLR, a multifactorial titration experiment was performed which co-titrated the amount of additional T7 RNA polymerase added into the reaction and the ratio of rUTP to RLR2b. For the experimental conditions a reaction volume of 60 μl was used instead of the 40 μl used for the standard cartridge assay. The performance of the different conditions was evaluated for cRNA yield and discrimination score after array hybridization. Each condition was performed with 6 arrays and the results were averaged over the 6 arrays. Similar results were observed for both the monolithic plate and the PEG arrays. Several conditions gave good performance.

Each reaction had 6 μl 10× IVT labeling buffer, 18 μl IVT labeling NTP mix, 6 μl IVT labeling enzyme mix, 5.8 μl RNase free water, 1 μl T7 RNA polymerase, 1.2 μl RLR2B (25 mM stock), 22 μl cDNA and RNase free water to 60 μl. Addition of 0, 1, 2, 3 or 4 extra μl of T7 polymerase and varying ratios of rUTP to RLR2B (5.19:2.31, 5.19:2.81 or 5.19:3.46) were tested. The best performance was seen with 1 extra μl T7 and 5.19:2.81, but other conditions also worked well. The final concentrations in the reaction are T7 RNA polymerase at 13.3 units/μl, RLR2B at 2.81 mM, rATP, rGTP, RCTP at 7.5 mM each and rUTP at 5.19 mM. The ratio of rUTP to RLR2B is 1.85:1. The T7 RNA polymerase is contributed both from the labeling enzyme mix and an additional boost of T7 (Ambion, catalog number 2085).

The hybridization conditions for the plate array were also optimized. In a preferred embodiment tetramethyl ammonium chloride (TMAC) is used to replace sodium chloride in the hybridization buffer. The following hybridization and wash conditions were tested: condition A: 1M Na in hybridization; 0.1M Na in Wash B (This is the standard condition employed in the 18 micron cartridge assay), condition B: 0.5M Na, 10% DMSO in hybridization; 0.1M Na in Wash B, condition C, 2.5M TMAC in hybridization; 0.1M Na in Wash B, condition D: 2M TMAC in hybridization; 0.1M Na in Wash B, condition E: 2.5M TMAC in hybridization; 0.2M Na in Wash B, and condition F: 0.5M Na, 15% DMSO in hybridization; 0.1M Na in Wash B. All the conditions (i.e. B, C, D, E, and F) gave satisfactory results in comparison to Condition A. In a preferred embodiment the hybridization conditions are 2.5 M TMAC, 100 mM MES, 20 mM EDTA and 0.01% Tween-20.

In a preferred embodiment after hybridization and washing the arrays are stored in a holding/scanning buffer. The holding/scanning buffer preferably stabilized probe/target hybrids. HFA scanning often requires long scan times, for example, scanning 96 HTA arrays on the Axon scanner may require an 8 hour scan. The holding/scanning buffer is designed to stabilize the array signal under un-refrigerated conditions. In a preferred embodiment the holding buffer is a MES based buffer. The standard cartridge assay uses an SSPE based buffer but the MES based buffer provides improved hybridization stability over time. The stability in the MES buffered holding solution promotes more increased stability, resulting in about 6% average signal loss over 6 hours at 30 C which is an improvement over the current standard holding buffer, “6×” SSPE which results in an average of >25% intensity loss at 30 C over 6 hours. The MES based holding buffer provides the necessary stability for HTA array to be stable for prolonged periods of incubation under normal scanning temperatures which may be close to ambient. In a preferred embodiment the array holding buffer is 68 mM MES, 1 M NaCl, and 0.01% Tween. After hybridization, washing and staining an array with hybridized target may be stored in an array holding buffer. The array holding buffer may be used for scanning as well. In a preferred embodiment the stringent wash buffer is 68 mM MES, 100 mM NaCl, and 0.01% Tween. 1.2× TMAC buffer in a preferred embodiment is 100 mM MES, 2.5 M TMAC, 20 mM EDTA and 0.01% Tween-20. To make 50 ml of 1.2× TMAC buffer mix 4.92 ml 12×MES, 30 ml 5 M TMAC, 2.4 ml 0.5 M EDTA, 0.06 ml 10% Tween and 12.62 ml DEPC water.

### EXAMPLE 1

Example 1 provides an example of an automated sample preparation protocol performed in 96 well plates. Each liquid transfer step may be performed by a liquid handling robot. Movement of plates may be performed by a plate handling robot. It should be understood that one or more steps may be performed manually as well.

For primer annealing mix 2 μl 50 μM T7-(dT)₂₄ and 8 μl water for each well and aliquot 10±1 μl per well in 96 well plates. About 5 μg total RNA is added to each well in 5 μl. Incubation after mixing is 10 min at 70° C. and 5 min at 4° C. For first strand cDNA synthesis cocktail mix 6 μl 5× 1st strand buffer, 3μ 0.1M DTT, 1.5 μl 10 mM dNTP mix, 1.5 μl SuperScript II and 3 μl water per well. Aliquot 15±1 μl per well in 96 well plates. Add 10 μl from primer annealing into each well and mix well. Incubate at 42° C. for 60 min and 4° C. for 5 min. For second strand cDNA mix per well 30 μl 5× 2nd strand buffer, 3 μl 10 mM dNTP mix, 1 μl 10 unit/ul DNA ligase, 4 μl 10 unit/ul DNA polymerase I and 1 μl 2 unit/μl Rnase H for a total volume of 39 μl. Aliquot 39±1.5 μl per well in 96 well plates. Transfer 91 μl water into each well of the first strand cDNA synthesis reaction, mix well and then transfer 111 μl μl to each well of the second strand cDNA synthesis plate (total in reaction is now 150 μl). Incubate at 16° C. for 120 minutes. For T4 DNA polymerase step mix 2 μl T4 DNA polymerase and 2 μl 1× T4 DNA polymerase buffer for each well and aliquot 4 μl into each well of a 96 well plate. Transfer 4 μl into each well of second strand synthesis reaction plate (total volume now 154 μl) and incubate 10 min at 16° C., 10 min at 72° C. and 5 min at 4° C. Transfer volume to wells of MinElute plate and treat according to manufacturers instructions (about 30 min under vacuum). Elute with 35 μl water, on orbital shaker at 1000 rpm for about 2 min. For IVT mix 6 μl 10×IVT buffer, 18 μl RLR labeling NTP mix (Affymetrix), 4 μl MegaShortScript and 10 μl RNAse-free water. Aliqout 38±1.5 μl per well into a 96 well plate. Add 35 μl eluted double stranded cDNA to each well and incubate at 37° C. for 4 hours. The product of the IVT reaction may be cleaned up using RNeasy MinElute (Qiagen). Elute in 45 μl water on orbital shaker at 1000 rpm for 2 min. Transfer entire eluate to cRNA concentration collector. To quantify yields measure OD(260) using 2 μl of eluate diluted into 198 μl water. After obtaining OD reading transfer 30 μl of eluate to each well of an equalization plate. Add water to each well based on the OD reading for that sample to obtain a concentration of about 0.625 μg/l. Preferably the RNA concentrations in the samples in a plate vary by less than 3%, 5% or 10% after normalization. Transfer 30 μl from equalization plate to each well of fragmentation plate. Fragmentation plate contains 7.5 μl 5× fragmentation buffer in each well. Mix well and incubate for 35 min at 94° C. and the 5 min at 4° C. The fragmented sample is now ready for hybridization to arrays and may be used for standard cartridge array hybridizations or hybridizations to arrays using peg array plates. During the sample preparation plates may be sealed with adhesive foil or another appropriate sealing material. Introduction of reagents may be by the use of a piercing device. If analyzing less than 96 samples, remaining wells may be filled with water. Volumes are provided on a per well basis. One of skill in the art will recognize that volumes are approximate and actual volumes are affected by carry over due to, for example, extra liquid coating the outside surface of a pipet tip or extra drops on the bottom of a pipet tip.

### EXAMPLE 2

Example 2 provides an example of a protocol for automated hybridization, washing and staining. Target for hybridization may be prepared according to the method disclosed in Example 1 or by manual methods.

Hybridization to array plates. Pre-hybe buffer is 1.1 μl 10 mg/ml HS DNA, 1.1 μl 50 mg/ml Acetylated BSA, 92 μl 1.2× Hyb Buffer (TMAC buffer) and 16 μl water per well. Transfer 100 μl to each well of the array plate (arrays present) and incubate at room temp for 10 min. Transfer 10 μl of fragmented cRNA from each well of the fragmentation plate into each well of the hybe plate containing in each well 3.02 μl 20× Bio B, C, D and Cre mix, 1.65 μl 3 nM B2 oligo, 1 μl HS DNA (10 mg/ml), 1 μl Ac BSA (50 mg/ml) and 83.33 μl 1.2× TMAC buffer per well. Mix well and incubate 5 min at 95° C. Remove the prehybe from the array plate and transfer 100 μl from the hybe plate to the array plate (arrays present) and incubate for 16 hours at 48° C. Wash at low stringency by removing hybridization sample volume and adding 200 μl 6× SSPE, incubate 1 min and remove and repeat with a new 200 μl 6× SSPE. Repeat the wash 3 to 7 times. Follow with a high stringency wash with 200 μl 0.1× MES, remove, and optionally repeat, then add a final 200 μl 0.1×MES and incubate at 48° C. for 40 min. Remove the MES buffer and add 100 μl of Stain 1. Stain 1 is 10 μl 2×MES buffer, 99 μl water, 8.8 μl Ac BSA, and 2.2 μl 1 mg/ml SAPE. Incubate at room temp for 12 min. Remove stain and transfer 100 μl 6× SSPE to each well. Incubate 1 min and remove 6× SSPE. Repeat wash step 14 more times. Remove final 6× SSPE and add 100 μl stain 2 to each well. Stain 2 is 110 μl 2× MES buffer, 97.5 μl water, 8.8 μl Ac BSA, and 2.2 μl 10 mg/ml Goat IgG and 1.32 μl 0.5 mg/ml biotinylated anti-strep Ab. Incubate for 20 min at room temp. Remove stain and wash with 200 μl 6× SSPE for 1 min. Repeat wash at least 5 more times. Remove final wash and add 100 μl stain 3 and incubate 15 min at room temp. Stain 3 is 110 μl 2× stain buffer, 99 μl water, 8.8 μl Ac BSA, and 2.2 μl 1 mg/ml SAPE. Wash 15 times with 200 μl 6× SSPET for 1 min each wash. Remove final wash and add 200 μl 0.1× MES holding buffer, remove, transfer 200 μl fresh 0.1×MES holding buffer to array plate, remove and add a final 200 μI fresh 0.1× MES holding buffer. The arrays are now ready for scanning. Scanning may be by the Axon scanner.

### EXAMPLE 3

Example 3 provides a second example of an automated sample preparation protocol performed in multi well plates. Each liquid transfer step may be performed by a liquid handling robot. Movement of plates may be performed by a plate handling robot. It should be understood that one or more steps may be performed manually as well.

For primer annealing mix 114 μl 50 μM T7-(dT)24 and 456 μl water and aliquot 5 μl per well in 96 well plates. About 5 μg total RNA is added to each well in 5 μl so the total is 10 μl in each well of the total RNA plate. Incubate after mixing for 10 min at 70° C. and 5 min at 4° C. For first strand cDNA synthesis cocktail mix 456 μl 5× 1st strand buffer, 228 μl 0.1 M DTT, 114 μl 10 mM dNTP mix, 114 μl SuperScript II and 228 μl water. Transfer 10 μl into each well of the total RNA plate 96. Incubate at 42° C. for 60 min and 4° C. for 5 min. For second strand cDNA mix per well 3150 μl 5× 2nd strand buffer, 315 μl 10 mM dNTP mix, 105 μl 10 unit/ul DNA ligase, 420 μl 10 unit/ul DNA polymerase 1 and 105 μl 2 unit/μl Rnase H for a total volume of 4095 μl. Transfer 91 μl water into each well of the total RNA plate and mix well. Transfer 39 μl second strand cDNA mix into each well of the total RNA plate and mix well. The total in each well of the total RNA plate is now 150 μl. Incubate at 16° C. for 120 minutes. For T4 DNA polymerase step mix 228 μl T4 DNA polymerase and 228 μl 1× T4 DNA polymerase buffer and aliquot 4 μl into each well of the total RNA plate and mix well. Incubate 10 min at 16° C., 10 min at 72° C. and 5 min at 4° C.

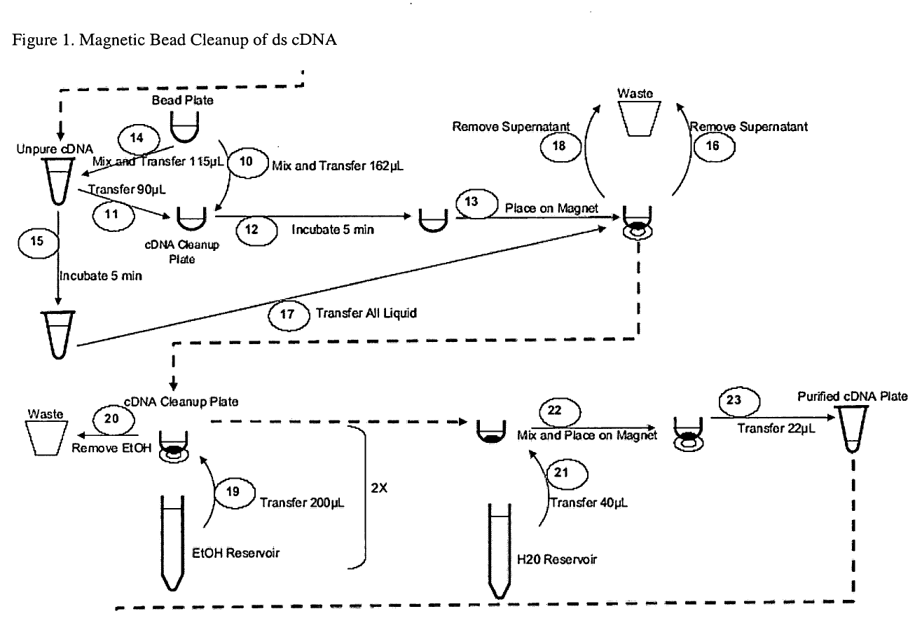

Magnetic bead cleanup of ds cDNA (see FIG. 1). Transfer 162 μl of mag bead solution (Agencourt) to the cDNA Cleanup Plate and transfer 90 μl of each double stranded cDNA reaction from the Total RNA Plate to each well of the cDNA Cleanup Plate and mix well. Incubate at room temp for 5 min to allow cDNA to bind to the beads. Move the cDNA Cleanup Plate to the magnet. Transfer 115 μl of magnetic beads into each well of the Total RNA Plate and incubate to bind cDNA to beads in the Total RNA Plate. Remove supernatant from cDNA Cleanup Plate on magnet. Transfer all liquid from the Total RNA Plate to the cDNA cleanup plate and incubate to capture the beads on the magnet, room temp for 10 min. Remove supernatant from cDNA Cleanup Plate on magnet. Wash beads twice with 200 μl EtOH, incubate 5 min at room temp to dry beads, add 40 μl water to beads and mix very well. Incubate 1 min at room temp. Move cDNA Cleanup Plate to magnet and incubate for 5 min at room temp to capture beads on magnet. Transfer 22 μl eluted cDNA to Purified cDNA Plate.

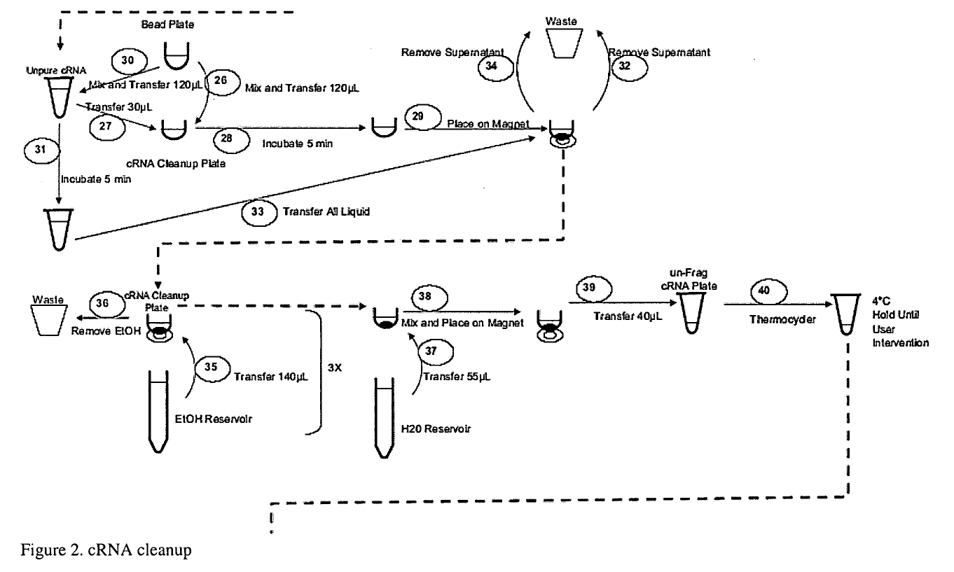

For IVT mix 630 μl 10×IVT buffer, 1890 μl RLR labeling NTP mix (HTA-RLR, Affymetrix), 630 μl Enzyme mix, 105 μl T7 RNA Pol (Ambion cat. 2085) and 735 μl RNAse-free water. Transfer 38 μl per well into each well of purified cDNA plate and incubate at 37° C. for 8 hours. The product of the IVT reaction may be cleaned up using Agencourt magnetic beads. See FIG. 2. Transfer 30 μl from purified cDNA plate (after IVT) to each well of cRNA cleanup plate. Transfer 120 μl mag beads to each well of cRNA cleanup plate and mix well and incubate 5 min at room temp. Remove cRNa cleanup plate to magnet. Transfer 120 μl magnetic beads to each well of purified cDNA plate and incubate to bind cRNA to beads in purified cDNA plate. Remove supernatant from cRNA cleanup plate on magnet. Transfer all liquied from purified cDNA plate to cRNA cleanup plate. Incubate to capture beads on magnet. Remove supernatant from cRNA cleanup plate on magenet. Wash beads 3 times with 140 μl EtOH. Incubate to dry beads. Add 55 μl water to beads mix well and incubate 5 min at room temp. Move cRNA cleanup plate to magnet. Incubate 5 min at room temp to capture beads on magnet. Transfer 40 μl eluted cRNA to un-Frag cRNA plate and put the plate at 4° C.

Transfer 198 μl water to Optical Plate 1 and transfer 2 μl from each well from the un-Frag cRNA plate to each well of the Optical Plate 1 and mix well. Take OD (260) reading. After obtaining OD reading transfer 35 μl of each cRNA sample to each well of an equalization plate. Add water to each well based on the OD reading for that sample to obtain a concentration of about 0.625 μg/μl. Preferably the RNA concentrations in the samples in a plate vary by less than 3%, 5% or 10% after normalization. Check OD again to confirm normalization.

Transfer 30 μl from equalization plate to each well of fragmentation plate. Fragmentation plate contains 7.5 μl 5× fragmentation buffer in each well. Mix well and incubate for 35 min at 94° C. and the 5 min at 4° C. The fragmented sample is now ready for hybridization to arrays and may be used for standard cartridge array hybridizations or hybridizations to arrays using peg array plates. During the sample preparation plates may be sealed with adhesive foil or another appropriate sealing material. Introduction of reagents may be by the use of a piercing device. If analyzing less than 96 samples, remaining wells may be filled with water. Volumes are provided on a per well basis. One of skill in the art will recognize that volumes are approximate and actual volumes are affected by carry over due to, for example, extra liquid coating the outside surface of a pipet tip or extra drops on the bottom of a pipet tip.

### EXAMPLE 4

Example 4 provides an example of a protocol for automated hybridization, washing and staining. Target for hybridization may be prepared according to the method disclosed in Example 1 or by manual methods.

Hybridization to array plates. For pre-hybe buffer mix 0.7 μl 10 mg/ml HS DNA, 0.7 μl 50 mg/ml Acetylated BSA, 58.6 μl 1.2× hybe buffer TMAC buffer and 10.19 μl water per well. Transfer 60 μl to each well of the pre-hybe tray and place peg array on prehybe tray. Transfer 10 μl of fragmented cRNA from each well of the fragmentation plate into each well of the hybe plate containing in each well 3.02 μl 20× Bio B, C, D and Cre mix, 1.65 μl 3 nM B2 oligo, 1 μl HS DNA (10 mg/ml), 1 μl Ac BSA (50 mg/ml) and 83.33 μl 1.2× TMAC buffer per well. Transfer the labeled cRNA in hybridization buffer to the Denatured Sample Plate, mix well and incubate 5 min at 95° C. Transfer 60 μl to Hybridization Tray and place the peg array on the Hybridization Tray. Place in hyb chamber and incubate for 16 hours at 48° C. In one aspect hybridization buffer is 100 mM MES, 1 M [Na+], 20 mM EDTA and 0.01% Tween-20.

Wash at low stringency by removing hybridization sample volume and adding 200 μl 6× SSPE, 0.01% Tween-20, incubate 1 min and remove and repeat with a new 200 μl 6× SSPE. Repeat the wash 3 to 7 times. Follow with a high stringency wash (HSW) with 68 mM Mes, 100 mM NaCl and 0.01% Tween-20, remove, and optionally repeat, then add a final 200 μl 0.1× MES and incubate at 48° C. for 40 min. Remove the MES buffer and add 70 μl of Stain 1. Stain 1 is 3783 μl 2×MES stain buffer, 3404.7 μl water, 302.6 μl Ac BSA, and 75.7 μl 1 mg/ml SAPE for 96 wells, 70 μl per well. Incubate at room temp for 12 min. Remove stain and transfer 100 μl 6× SSPE to each well. Incubate 1 min and remove 6× SSPE. Repeat wash step 14 more times. Remove final 6× SSPE and add 70 μl stain 2 to each well. Stain 2 is 3783 μl 2×MES buffer, 3359.35 μl water, 302.64 μl Ac BSA, and 75.66 μl 10 mg/ml Goat IgG and 45.40 μl 0.5 mg/ml biotinylated anti-strep Ab for 96 wells. Incubate for 20 min at room temp. Remove stain and wash with 200 μl 6× SSPE for 1 min. Repeat wash at least 5 more times. Remove final wash and add 70 μl stain 3 and incubate 15 min at room temp. Stain 3 is 3783 μl 2×MES stain buffer, 3783 μl water, 302.6 μl Ac BSA, and 75.7 μl 1 mg/ml SAPE for 96 wells. Wash 15 times with 200 μl 6× SSPET for 1 min each wash. Remove final wash and add 200 μl MES holding buffer which is 100 mM MES, 5 M NaCl and 0.01% Tween 20, remove, transfer 200 μl fresh MES holding buffer to array plate, remove and add a final 200 μl fresh MES holding buffer. The arrays are now ready for scanning. Scanning may be by the Axon scanner.

### EXAMPLE 5

Example 1 provides an example of an automated sample preparation protocol performed in 96 well plates. Each liquid transfer step may be performed by a liquid handling robot. Movement of plates may be performed by a plate handling robot. It should be understood that one or more steps may be performed manually as well.

For primer annealing mix 1 μl 50 μM T7-(dT)24 and 4 μl water for each well and aliquot 5±1 μl per well in 96 well plates. About 5 μg total RNA is added to each well in 5 μl. Incubation after mixing is 10 min at 70° C. and 5 min at 4° C. For first strand cDNA synthesis cocktail mix 6 μl 5× 1st strand buffer, 3μ 0.1M DTT, 1.5 μl 10 mM dNTP mix, 1.5 μl SuperScript II and 3 μl water per well. Aliquot 15±1 μl per well in 96 well plates. Add 10 μl from primer annealing into each well and mix well. Incubate at 42° C. for 60 min and 4° C. for 5 min. For second strand cDNA mix per well 30 μl 5× 2nd strand buffer, 3 μl 10 mM dNTP mix, 1 μl 10 unit/μl DNA ligase, 4 μl 10 unit/μl E. coli DNA polymerase 1 and 1 μl (2 unit/μl) RNase H for a total volume of 391 μl. Aliquot 39±1.5 μl per well in 96 well plates. Transfer 91 μl water into each well of the first strand cDNA synthesis reaction, mix well and then transfer 111 μl to each well of the second strand cDNA synthesis plate (total in reaction is now 150 μl). Incubate at 16° C. for 120 minutes. For T4 DNA polymerase step mix 2 μl T4 DNA polymerase and 2 μl 1× T4 DNA polymerase buffer for each well and aliquot 4±1 μl into each well of a 96 well plate. Transfer 4 μl into each well of second strand synthesis reaction plate (total volume is now ˜154 μl) and incubate 10 min at 16° C., 10 min at 72° C. and 5 min at 4° C. Transfer volume to wells of MinElute plate and treat according to manufacturers instructions (about 30 min under vacuum). Elute with 35 μl water, on orbital shaker at 1000 rpm for about 2 min. For IVT mix 6 μl 10× IVT buffer, 18 μl RLR labeling NTP mix (Affymetrix), 6 μl labeling enzyme mix, 1 μl T7 RNA Polymerase and 7 μl RNase-free water. Aliqout 38±1.5 μl per well into a 96 well plate. Add 35 μl eluted double stranded cDNA to each well and incubate at 37° C. for 4 hours. The product of the IVT reaction may be cleaned up using RNeasy MinElute (Qiagen). Elute in 45 μl water on orbital shaker at 1000 rpm for 2 min. Transfer entire eluate to cRNA concentration collector. To quantify yields measure OD(260) using 2 μl of eluate diluted into 198 μl water. After obtaining OD reading transfer 30 μl of eluate to each well of an equalization plate. Add water to each well based on the OD reading for that sample to obtain a concentration of about 0.625 μg/l. Preferably the RNA concentrations in the samples in a plate vary by less than 3%, 5% or 10% after normalization. Transfer 30 μl from equalization plate to each well of fragmentation plate.

Fragmentation plate contains 7.5 μl 5× fragmentation buffer in each well. Mix well and incubate for 35 min at 94° C. and the 5 min at 4° C. The fragmented sample is now ready for hybridization to arrays and may be used for standard cartridge array hybridizations or hybridizations to arrays using peg array plates. During the sample preparation plates may be sealed with adhesive foil or another appropriate sealing material. Introduction of reagents may be by the use of a piercing device. If analyzing less than 96 samples, remaining wells may be filled with water. Volumes are provided on a per well basis. One of skill in the art will recognize that volumes are approximate and actual volumes are affected by carry over due to, for example, extra liquid coating the outside surface of a pipet tip or extra drops on the bottom of a pipet tip.

### CONCLUSION

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

We claim:

1. A automated method for preparing a plurality of targets for hybridization to a microarray comprising:
incubating nucleic acid probes in a hybridization buffer so that the nucleic acid probes bind to the surface of a substrate, wherein the hybridization buffer comprises tetramethyl ammonium chloride (TMAC).

2. The method of claim 1, wherein the hybridization buffer further comprises MES, EDTA and Tween 20.

3. The method of claim 1, wherein nucleic acid comprises of cRNA or cDNA

4. The method of claim 1, wherein the concentration of TMAC is between about 1 M to about 4M.

5. The method of claim 1, wherein the concentration of MES is between about 50 mM and 200 mM.

6. The method of claim 1, wherein the concentration of EDTA is between 5 mM and 40 mM.

6. The method claim 1, wherein the concentration of Tween is between 0.001% and 0.5%.

7. The method of claim 1, wherein the incubation temperature is about 40 to 55° C.

8. The method of claim 1, wherein the incubation time is between 10 and 20 hours.

9. A microarray hybridization buffer comprising betwee 75 and 150 mM MES, between 15 and 30 mM EDTA, between 0.001 and 0.02% Tween 20, and between 2 and 3 M TMAC and optionally comprising herring sperm DNA, acetylated BSA, Denhardt's solution and human cot-1 DNA.

10. An array holding buffer comprising about 60 to 80 mM MES, about 0.8 to 1.2 M NaCl, and about 0.005 to 0.02% Tween.

11. A method for preparing amplified and labeled cRNA from a plurality of RNA samples in parallel comprising:
synthesizing first strand cDNA from the RNA using reverse transcriptase and a T7 promoter primer;
synthesizing second strand cDNA using a DNA polymerase and RNase H to obtain double stranded cDNA with a T7 RNA polymerase promoter;
cleaning the double stranded cDNA using solid phase reversible immobilization to magnetic beads;
eluting the cleaned double stranded cDNA from the magnetic beads; and
mixing the cleaned double stranded cDNA in a reaction comprising T7 RNA polymerase and labeled nucleotides to generate cRNA.

12. The method of claim 11 wherein at least 8 samples are analyzed.

13. The method of claim 11 wherein at least 24 samples are analyzed.

14. The method of claim 11 wherein at least 96 samples are analyzed.

15. The method of claim 11 wherein the cRNA is labeled with biotin.

16. The method of claim 11 wherein the samples are processed on an automated liquid handling robot.
